(19) United States
(12) Patent Application Publication
(10) Pub. No.: US 2012/0135465 A1
(43) Pub. Date: May 31, 2012

(54) Endoglucanase For Reducing The Viscosity Of A Plant Materials Slurry

(76) Inventors: Martien H. Bergsma, Zoetemeer (NL); Gerhard Konieczny-Janda, Pattensen (DE); Jayarama K. Shetty, Pleasanton, CA (US); Paula Johanna Maria Teunissen, Saratoga, CA (US); Jan Hendrik Van Tuijl, Uihoorn (NL)

(21) Appl. No.: 13/263,314

(22) PCT Filed: Apr. 8, 2010
(86) PCT No.: PCT/US10/30430

§ 371 Date: Feb. 16, 2012

Related U.S. Application Data
(60) Provisional application No. 61/167,617, filed on Apr. 8, 2009.

(51) Int. Cl. C12P 19/00; C12N 9/42

(52) U.S. Cl. 435/72; 435/209

(57) ABSTRACT

The present disclosure relates to composition comprising EG cellulase and methods of use, thereof. The compositions are useful, e.g., for reducing the viscosity of plant material slurry.

### PRIORITY

The present application claims priority to U.S. Provisional Application Ser. No. 61/167,617, filed on Apr. 8, 2009, which is hereby incorporated by reference in its entirety.

### TECHNICAL FIELD

The present disclosure relates to compositions comprising endoglucanase (EG) cellulase and methods of use, thereof. The compositions are useful, e.g., for reducing the viscosity of plant material compositions, such as barley mash.

### BACKGROUND

Cellulose and hemicellulose are the most abundant plant materials produced by photosynthesis. They can be degraded and used as an energy source by numerous microorganisms (e.g., bacteria, yeast and fungi) that produce extracellular enzymes capable of hydrolysis of the polymeric substrates to monomeric sugars (Aro et al., J. Biol. Chem., 276:24309-14, 2001).

Cellulases are enzymes that hydrolyze cellulose (β-1,4-glucan or β-D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and (β-glucosidases (β-D-glucoside glucohydrolase; EC 3.2.1.21) (“BG”). (Knowles et al., TIBTECH 5:255-61, 1987; and Schulein, Methods Enzymol., 160:234-43, 1988).

Endoglucanases act mainly on the amorphous parts of the cellulose fibre to hydrolyze internal β-1,4-glucosidic bonds in regions of low crystallinity. Cellobiohydrolases hydrolyze cellobiose from the reducing or non-reducing end of cellulose and are able to degrade crystalline cellulose (Nevalainen and Penttila, The Mycota Vol. III, pp. 303-19, 1995). The presence of a cellobiohydrolase (CBH) in a cellulase system is believed to be required for efficient solubilization of crystalline cellulose (Suurnakki et al., Cellulose 7:189-209, 2000). β-glucosidase acts to liberate D-glucose units from cellobiose, cello-oligosaccharides, and other glucosides (Freer, J. Biol. Chem., 268:9337-42, 1993). β-glucosidases have also been shown to catalyze the hydrolysis of alkyl and/or aryl beta-D-glucosides such as methyl β-D-glucoside and p-nitrophenyl glucoside as well as glycosides containing only carbohydrate residues, such as cellobiose. This yields glucose as the sole product for the microorganism and reduces or eliminates cellobiose which inhibits cellobiohydrolases and endoglucanases.

Cellulases are known to be produced by a large number of bacteria, yeast and fungi. Certain fungi produce complete cellulase systems that include exo-cellobiohydrolases or CBH-type cellulases, endoglucanases or EG-type cellulases and β-glucosidases or BG-type cellulases. Other fungi and bacteria express little or no CBH-type cellulases. Generally, it is believed that the EG components and CBH components must interact synergistically to efficiently degrade cellulose.

The fungal cellulase classifications of CBH, EG and BG can be further expanded to include multiple components within each classification. For example, multiple CBHs, EGs and BGs have been isolated from a variety of fungal sources including Trichoderma reesei (also referred to as Hypocrea jecorina), which contains known genes for two CBHs, i.e., CBH I (“CBH1”) and CBH II (“CBH2”), at least eight EGs, i.e., EG I, EG II, EG III, EG IV, EG V, EG VI, EG VII, and EG VIII, and at least five BGs, i.e., BG 1, BG 2, BG 3, BG4 and BG 5. EG IV, EG VI, and EG VIII also have xyloglucanase activity.

Cellulases are useful commercially in the degradation of cellulase biomass for use by microorganisms, e.g., for ethanol production. In addition to hydrolyzing cellulase to saccharides that can be metabolized by microorganism, cellulases reduce the viscosity of plant slurries to allow them to be efficiently mixed and transferred. It was heretofore believed that combinations of cellulases, including endoglucanases and cellobiohydrolases had the greatest affect on viscosity, and that mixtures of cellulases were required for efficient viscosity reduction of plant material slurries.

Although compositions for reducing the viscosity of plant material slurries have been previously described, there remains a need for new and improved cellulase compositions, preferably with defined components.

### SUMMARY

The present compositions and methods relate to the use of endoglucanase (EG) to reduce the viscosity of plant material slurries.

In one aspect, a method for reducing the viscosity of a plant material slurry is provided, comprising adding to the slurry a composition comprising an isolated endoglucanase (EG) cellulase. In some embodiments, the composition is substantially free of other cellulases. In some embodiments an addition cellulase is separately added to the slurry but is not required to reduce the viscosity of the slurry.

In a related aspect, a method for reducing the viscosity of a plant material slurry is provided, comprising adding to the slurry a composition consisting essentially of endoglucanase (EG) cellulase.

In another aspect, a method for reducing the viscosity of a plant material slurry is provided, comprising adding to the slurry a single-cellulase composition comprising endoglucanase (EG) cellulase. In some embodiments, an addition cellulase is separately added to the slurry but is not required to reduce the viscosity of the slurry.

In some embodiments, the EG cellulase is expressed in a filamentous fungus. In some embodiments, the EG cellulase is expressed in Trichoderma reesei. In some embodiments, the EG cellulase is expressed under control of the cbh1 promoter.

In some embodiments, the EG cellulase is purified to at least 70% of total protein in the composition. In some embodiments, the EG cellulase is purified to at least 80% of total protein in the composition. In some embodiments, the EG cellulase is purified to at least 90% of total protein in the composition. In some embodiments, the EG cellulase is purified to at least 95% of total protein in the composition. In some embodiments, the EG cellulase is purified to at least 97% of total protein in the composition.

In some embodiments, the EG cellulase is selected from the group consisting of EG I, EG II, and EG III. In some embodiments, the EG cellulase is EG II.

In some embodiments, the EG cellulase is a Trichoderma reesei (Hypocrea jecorina) EG cellulase. In particular embodiments, the EG cellulase is a T. reesei EG II cellulase.

In some embodiments, the reduction in viscosity as a result of adding the EG cellulase is at least equivalent to a reduction in viscosity as a result of adding a mixture of cellulases wherein EG is a component. In some embodiments, the mixture of cellulases includes at least one CBH cellulase, BG cellulase, and/or xylanase.

In some embodiments, reducing the viscosity of the slurry is performed at a temperature greater than about 65° C. In some embodiments, reducing the viscosity of the slurry is performed at a temperature not less than about 65° C. In particular embodiments, reducing the viscosity of the slurry is performed at a temperature of from about 65° C. to about 75° C.

In some embodiments, EG is added to a slurry prior to boiling. In some embodiments, the addition of EG follows boiling the slurry.

In some embodiments, viscosity in the plant material slurry is primarily due to the presence of betaglucan. In some embodiments, the plant material is from barley or oats. In particular embodiments, the plant material is from barley.

In a related aspect, a single-cellulase composition comprising EG cellulase is provided for use as described. In particular embodiments, the EG cellulase is expressed as a secreted polypeptide in filamentous fungi wherein other cellulase genes are deleted or disrupted.

In some embodiments, the EG cellulase is selected from the group consisting of EG I, EG II, and EG III. In some embodiments, the EG cellulase is EG II. In particular embodiments, the EG cellulase is a Trichoderma reesei (Hypocrea jecorina) EG II cellulase.

In some embodiments, the single-cellulase composition does not comprise a CBH cellulase, a BG cellulase, and/or a xylanase.

In a related aspect, a cellulase composition is provided, comprising one or more EG cellulases in the absence of a CBH cellulase, a BG cellulase, and/or a xylanase, for use in reducing the viscosity of a plant material slurry in which the viscosity in the plant material slurry is primarily due to the presence of betaglucan.

Other aspects and embodiments will be apparent from the following detailed description.

### BRIEF DESCRIPTION OF THE DRAWINGS

FIG. 1 is an image of a Coomassie-stained SDS-PAGE gel showing the polypeptides present in an EG II preparation and in the commercial product OPTIMASH™ BG.

FIGS. 2A and 2B are graphs comparing the ability of EG II (EG 2) and OPTIMASH™ BG to reduce the viscosity of a wheat composition in a low temperature process (2A) and in a conventional liquefaction process (2B).

FIG. 3 is a graph comparing the ability of EG II and OPTIMASH™ BG (BG) to reduce the viscosity of a barley composition.

FIG. 4 is a graph comparing the ability of EG II and OPTIMASH™ BG (BG) to reduce the viscosity of a barley composition. The enzymes were added at the slurry make-up step (A S/M) with and without additional enzyme being added following the boiling step (A Boiling).

FIG. 5 is a graph comparing the ability of EG II and OPTIMASH™ BG (BG) to reduce the viscosity of a barley composition. The enzymes were added before the third liquefaction step.

FIGS. 6A and 6B are graphs showing the pH/temperature activity profiles of OPTIMASH™ BG (5A) and EG II (5B).

FIG. 7 is an image of a Coomassie-stained SDS-PAGE gel showing the polypeptides present in EG I and EG III preparations.

FIG. 8 is a graph comparing the ability of EG I, EG II, and EG III to reduce the viscosity of a barley composition.

FIG. 9A is the amino acid sequence of an exemplary EG II polypeptide. FIG. 9B is the nucleotide sequence encoding the exemplary EG II polypeptide.

FIG. 10A is the amino acid sequence of an exemplary EGI polypeptide. FIG. 10B is the amino acid sequence of an exemplary EG III polypeptide.

### DETAILED DESCRIPTION

### I. Introduction

The present compositions and methods relate to the use of endoglucanase (EG) for reducing the viscosity of plant material compositions (i.e., slurries). The compositions and methods find application in grain processing, where cellulase components present in plant materials result in viscous slurries that are difficult to mix, transfer, filter, or otherwise manipulate.

Previous compositions and methods for reducing the viscosity of plant material slurries relied on a combination of cellulases, typically being provided in a largely undefined mixture of cellulases obtained from one of more organisms, such as a filamentous fungus. Exemplary compositions include OPTIMASH™ BG (Danisco, Genencor Division, Palo Alto, Calif., USA), a crude secreted protein product obtained from Trichoderma reesei that includes EG cellulases in combination with other types of cellulases.

The present compositions and methods are based on the unexpected finding that a substantially purified preparation of EG, in the absence of other cellulases, can reduce the viscosity of some plant material compositions at least as efficiently as conventional mixtures of cellulases. Notably, EG is effective in reducing the viscosity of plant materials in which viscosity is primary due to the presence of betaglucan, as found in, e.g., barley and oat slurries.

The ability to use a substantially purified preparation of EG to reduce the viscosity of a plant material slurry avoids the addition of unnecessary cellulases and other, partially characterized or even unknown proteins into the slurry, which material may adversely affect downstream processing or fermentation. The use of a substantially purified EG preparation also allows higher specific activity, since only the most important cellulase is present in the viscosity lower composition. These and other features of the compositions and methods will be apparent from the following description and examples.

### II. Definitions

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used herein. Practitioners are particularly directed to Sambrook et al., MOLECULAR CLONING: A LABORATORY MANUAL (Second Edition), Cold Spring Harbor Press, Plainview, N.Y., 1989, and Ausubel F M et al., Current Protocols in Molecular Biology, John Wiley & Sons, New York, N.Y., 1993, for definitions and terms of the art. Numeric ranges are inclusive of the numbers defining the range.

The following terms are defined for clarity:

As used herein, the term “polypeptide” refers to a compound made up of a chain of amino acid residues linked by peptide bonds. Unless otherwise indicated, the term “protein” is synonymous with the term “polypeptide.”

As used herein, a “native” polypeptide is one found naturally occurring in nature.

As used herein, a “variant” polypeptide is derived from a native polypeptide by addition of one or more amino acids to either or both the C and N-terminal end; substitution of one or more amino acids at one or a number of different sites in the amino acid sequence; deletion of one or more amino acids at either or both ends of the protein or at one or more sites in the amino acid sequence; changing the charge of an amino acid; chemically modifying a polypeptide; or combinations, thereof. The preparation of a polypeptide variant may be performed by any means know in the art, including modifying a nucleic acid sequence that encodes a polypeptide, chemical modification of a polypeptide or amino acids for incorporation into a polypeptide, and the like.

As used herein, the term “nucleic acid molecule” includes DNA, RNA, and cDNA molecules, and their derivatives, whether obtained from an organism or synthesized in a laboratory. It will be understood that, as a result of the degeneracy of the genetic code, a multitude of nucleotide sequences encoding a given polypeptide may exist.

As used herein, a “heterologous” nucleic acid construct or sequence has a portion of the sequence which is not native to the cell in which it is expressed. Heterologous, with respect to a control sequence refers to a control sequence (i.e., promoter or enhancer) that does not function in nature to regulate the expression of a subject gene. Generally, heterologous nucleic acid sequences are not endogenous to the cell or part of the genome in which they are present, and have been added to the cell, by infection, transfection, transformation, microinjection, electroporation, or the like. A “heterologous” nucleic acid construct may contain a control sequence/DNA coding sequence combination that is the same as, or different from a control sequence/DNA coding sequence combination found in the native cell.

As used herein, the term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

As used herein, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

As used herein, the term “plasmid” refers to a circular double-stranded (ds) DNA construct used as a cloning vector, and which forms an extrachromosomal self-replicating genetic element in many bacteria and some eukaryotes.

As used herein, the term “selectable marker-encoding nucleotide sequence” refers to a nucleotide sequence which is capable of expression in cells and where expression of the selectable marker confers to cells containing the expressed gene the ability to grow in the presence of a corresponding selective agent, or under corresponding selective growth conditions.

As used herein, the term “promoter” refers to a nucleic acid sequence that functions to direct transcription of a downstream gene. The promoter will generally be appropriate to the host cell in which the target gene is being expressed. The promoter, together with other transcriptional and translational regulatory nucleic acid sequences (also termed “control sequences”), are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

As used herein, the terms “chimeric gene” or “heterologous nucleic acid construct” refers to a non-native gene (i.e., one that has been introduced into a host) that may be composed of parts of different genes, including regulatory elements. A chimeric gene construct for transformation of a host cell is typically composed of a transcriptional regulatory region (promoter) operably linked to a heterologous protein coding sequence, or, in a selectable marker chimeric gene, to a selectable marker gene encoding a protein conferring, for example, antibiotic resistance to transformed cells. A typical chimeric gene of the present invention, for transformation into a host cell, includes a transcriptional regulatory region that is constitutive or inducible, a protein coding sequence, and a terminator sequence. A chimeric gene construct may also include a second DNA sequence encoding a signal peptide if secretion of the target protein is desired.

As used herein, nucleic acid sequences are “operably linked” when one nucleic acid sequences is placed in a functional relationship with another nucleic acid sequence. For example, DNA encoding a secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors, linkers or primers for PCR are used in accordance with conventional practice.

As used herein, the term “gene” means the segment of DNA involved in producing a polypeptide chain, that may or may not include regions preceding and following the coding region, e.g., 5′ untranslated (5′ UTR) or “leader” sequences and 3′ UTR or “trailer” sequences, as well as intervening sequences (introns) between individual coding segments (exons).

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

As used herein, the terms “transformed,” “stably transformed,” or “transgenic,” with reference to a cell, means that the cell has a non-native (heterologous) nucleic acid sequence integrated into its genome or has an episomal plasmid that is maintained through multiple generations.

As used herein, the term “expression” refers to the process by which a polypeptide is produced by a host organism based on the nucleic acid sequence of a gene. The process includes both transcription and translation. The polypeptide may remain in the host cell or be secreted into the surrounding medium.

As used herein, the term “introduced” in the context of inserting a nucleic acid sequence into a cell, means “transfection,” “transformation,” or “transduction,” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (e.g., transfected mRNA).

As used herein, the term “betaglucan” refers to a branched polysaccharide that includes primarily 1,3 and 1,4 glycosidic bonds. Typically groups of two to four 1,4-betaglucan units are linked by single 1,3 linkages. Betaglucans are present in, e.g., the bran of cereal grains and the cell walls of fungi and some bacteria. Betaglucans are most abundant in barley and oats and less abundant in rye and wheat.

As used herein, “pentosans” (also called “arabinoxylans” or “hemicellulose”) are a mixture of non-cellulosic polymers that may include pentoses, hexoses, side chains, phenolics, and even proteins. Exemplary sugars present in pentosans include D-galactose, D-glucose, L-arabinose, D-xylose, D-glucuronic acid, and 4-O-methyl-glucuronic acid. Pentosans are often gummy components with high water binding capacity. The presence of pentosans in plant material compositions leads to increased viscosity, which can interfere with filtration, mixing, and handling.

As used herein, the terms “cellulase,” “cellulolytic enzyme,” or “cellulase enzyme” refer to a category of enzymes capable of hydrolyzing cellulose polymers to shorter cello-oligosaccharide oligomers, cellobiose and/or glucose. Numerous examples of cellulases, such as exoglucanases, exocellobiohydrolases, endoglucanases, and glucosidases have been obtained from cellulolytic organisms, particularly including fungi, plants and bacteria. The enzymes made by these microbes are mixtures of proteins with three types of actions useful in the conversion of cellulose to glucose: endoglucanases (EG), cellobiohydrolases (CBH), and beta-glucosidase. These three different types of cellulase enzymes act synergistically to convert cellulose and its derivatives to glucose.

As used herein, “endoglucanase” or “EG” is used to refer to a group of polypeptides in the family EC 3.2.1.4 that are characterized by the presence of a cellulose binding domain and their ability to hydrolysis 1,4-β-D-glycosidic linkages in cellulose.

As used herein, the term “cellulose binding domain” refers to portion of the amino acid sequence of a cellulase or a region of the enzyme that is involved in the cellulose binding activity of a cellulase or derivative thereof. Cellulose binding domains generally function by non-covalently binding the cellulase to cellulose, a cellulose derivative or other polysaccharide equivalent thereof. Cellulose binding domains permit or facilitate hydrolysis of cellulose fibers by the structurally distinct catalytic core region, and typically function independent of the catalytic core. Thus, a cellulose binding domain will not possess the significant hydrolytic activity attributable to a catalytic core. In other words, a cellulose binding domain is a structural element of the cellulase enzyme protein tertiary structure that is distinct from the structural element which possesses catalytic activity. Cellulose binding domain and cellulose binding module may be used interchangeably herein.

As used herein, the term “signal sequence” refers to a sequence of amino acids at the N-terminal portion of a protein that facilitates the secretion of the mature form of the protein outside the cell. The mature form of the extracellular protein lacks the signal sequence that is cleaved off during the secretion process.

As used herein, the term “host cell” refers to a cell that contains a vector and supports the replication, and/or transcription or transcription and translation (expression) of an expression construct. Host cells may be prokaryotic cells, such as E. coli, or eukaryotic cells such as yeast, plant, insect, amphibian, or mammalian cells. Exemplary host cells are filamentous fungi.

As used herein, the term “filamentous fungi” means any and all filamentous fungi recognized by those of skill in the art. A preferred fungus is selected from the group consisting of Aspergillus, Trichoderma, Fusarium, Chrysosporium, Penicillium, Humicola, Neurospora, or alternative sexual forms thereof such as Emericella, Hypocrea. It has now been demonstrated that the asexual industrial fungus Trichoderma reesei is a clonal derivative of the ascomycete Hypocrea jecorina (See, Kuhls et al., Proc. Nat'l. Acad. Sci. U.S.A., 93:7755-60, 1996).

As used herein, the term “surfactant” refers to any compound generally recognized in the art as having surface active qualities. Thus, for example, surfactants comprise anionic, cationic and nonionic surfactants such as those commonly found in detergents. Anionic surfactants include linear or branched alkylbenzenesulfonates; alkyl or alkenyl ether sulfates having linear or branched alkyl groups or alkenyl groups; alkyl or alkenyl sulfates; olefinsulfonates; and alkanesulfonates. Ampholytic surfactants include quaternary ammonium salt sulfonates, and betaine-type ampholytic surfactants. Such ampholytic surfactants have both the positive and negative charged groups in the same molecule. Nonionic surfactants may comprise polyoxyalkylene ethers, as well as higher fatty acid alkanolamides or alkylene oxide adduct thereof, fatty acid glycerine monoesters, and the like.

As used herein, the term “detergent composition” refers to a mixture which is intended for use in a wash medium for the laundering of soiled cellulose containing fabrics. In the context of the present invention, such compositions may include, in addition to cellulases and surfactants, additional hydrolytic enzymes, builders, bleaching agents, bleach activators, bluing agents and fluorescent dyes, caking inhibitors, masking agents, cellulase activators, antioxidants, and solubilizers.

As used herein, the terms “substantially isolating” and “substantially purifying” an expressed polypeptide generally mean separating an expressed polypeptide from other cellular or media components, or other components with which it is naturally associated, such that the expressed polypeptides represent at least 70%, at least 80%, preferably at least 90%, and even at least 96%, at least 97%, at least 98%, or at least 99% (wt/wt) of the total protein present in a composition. Such compositions may be referred to as “substantially pure” or may be said to include “substantially a single polypeptide.” Such separation may be performed using column chromatography (including affinity chromatography) and/or other biochemical techniques known in the art.

As used herein, a composition is “enriched” for a polypeptide if it has been processed to include a greater proportion of the polypeptide than would be found without processing. As used herein, enrichment encompasses “isolation” and “purification” but does not require the same level of isolation. For example, where a starting material includes only 10% of a subject polypeptide, an enriched composition may comprise any amount that is greater than 10%. In some cases, an enriched composition includes at least 50% (wt/wt) of a subject protein, or even at least 60%, 70%, or more.

As used herein, a “single cellulase composition” refers to a substantially isolated or purified EG cellulase-containing composition that does not require the presence of another cellulase (other than EG) to reduce the viscosity of a plant material slurry as described, herein. A single cellulase composition is distinct from an enriched cellulase composition, or a mixed cellulase composition, which may rely on the activities of more than one type of cellulase to reduce the viscosity of a plant material slurry. A single cellulase composition may be produced by expressing one or more EG cellulases in a host cell that does not express other cellulases. Proteins other than EG cellulases may be present in any amounts without defeating the definition of a single-cellulase composition. In the present context, a feature of the described single-cellulase EG compositions is that they do not rely on CBH-type cellulase activity, BG-type cellulase activities, and/or xylanase activity, to liquify barley (or related) slurries.

As used herein, a composition is “substantially free” of specified other components if the other components are present at an undetectable level, or present at a level such that they do not contribute to the specified enzymatic process performed by the composition. For example, the present EG compositions are substantially free of CBH-type cellulases, BG-type cellulases, and/or xylanases, if CBH-type cellulases, BG-type cellulases, and/or xylanases, are undetectable in the EG compositions, or if CBH-type cellulases, BG-type cellulases, and/or xylanases are present in such low levels that they do not exhibit a detectable amount of activity. Such EG compositions may be described as “consisting essentially of EG,” since EG is the only essential cellulase present in the composition.

As used herein, the terms “active” and “biologically active” refer to a biological activity associated with a particular polypeptide. For example, the enzymatic activity associated with a cellulase is the ability to hydrolyze cellulose to glucose, cellobiose, cellooligosaccharides, and the like.

As used herein, a “wild type” cellulase is one having the same amino acid sequence as one produced by a naturally occurring organism.

As used herein, the term “slurry” refers to a composition comprising plant materials mixed with a liquid to form a mixture, suspension, solution, or combinations, thereof, with respect to components of the plant material, including cellulosic components. The liquid is preferably water but may include salts, surfactants, minerals, co-factors, buffers, and the like. Exemplary slurries include grain mash or wort, fermentation broth, suspended waste grain and other cellulose-rich materials, and the like.

The use of the singular includes the plural unless specifically stated otherwise. The use of “or” means “and/or” unless otherwise stated or apparent from context. Likewise, the terms “comprise,” “comprising,” “comprises,” “include,” “including” and “includes” are not intended to be limiting. All patents and publications, including all amino acid and nucleotide sequences disclosed within such patents and publications, referred to herein are expressly incorporated by reference. Unless otherwise indicated, nucleic acids are written left to right in 5′ to 3′ orientation and amino acid sequences are written left to right in amino to carboxyl orientation. Paragraph headings are provided to assist the reader and are not intended as limitations. Aspects or embodiments described under a particular heading may apply to the specification as a whole.

It is to be understood that both the foregoing general description and the following detailed description are exemplary and explanatory and are not intended to be restrictive of the compositions and methods described herein. The practice of the present compositions and methods will employ, unless otherwise indicated, conventional techniques of molecular biology, microbiology, recombinant DNA, and immunology, which are within the skill of the art. Such techniques are explained fully in the literature. See, e.g., Sambrook et al., 1989; Freshney, Animal Cell Culture, 1987; Ausubel et al., 1993; and Coligan et al., Current Protocols in Immunology, 1991. All reference cited herein are expressly incorporated by reference in their entirety.

### III. EG Cellulase

The endoglucanase (EG) cellulases are a group of polypeptides in the family EC 3.2.1.4 that are characterized by the presence of a cellulose binding domain and their ability to hydrolysis 1,4-β-D-glycosidic linkages in cellulose. EG cellulases for use in the present compositions, and methods for their cloning, expression, and isolation, are discussed, below.

A. EG Polypeptides

The amino acid sequences of a number of EG enzymes have been described and are available through public databases, such as Genbank. Exemplary amino acid sequences are found in the Genbank entries identified in Table 1-3, below:

TABLE 1
Exemplary EG I amino acid sequences

| Organism | Genbank Accession No. |
|---|---|
| Rhizopus stolonifer | gi267712097 |
| Talaromyces emersonii | gi21264637 |
| Thermoascus aurantiacus | gi24942374, gi16356671, gi61676026, |
| | gi16356671 |
| Ralstonia solanacearum | gi219903548, gi219903545, gi219903543, |
| | gi219903538, gi219903536, gi219903534, |
| | gi219903532, gi219903530, gi219903528, |
| | gi219903526, gi219903524, gi219903522, |
| | gi219903520 |
| Dimocarpus longan | gi254031737 |
| Volvariella volvacea | gi189498328 |
| Trichoderma viride | gi183228137 |

TABLE 2
Exemplary EG II amino acid sequences

| Organism | Genbank Accession No. |
|---|---|
| Trichoderma reesei | gi121794, gi77176916 |
| (Hypocrea jecorina) | |
| Trichoderma viride | gi4062993 |
| Penicillium janthinellum | gi984166 |
| Penicillium decumbens | gi163644901 |
| Volvariella volvacea | gi49333361 |
| Sclerotinia sclerotiorum | gi121792 |
| Pyrenophora tritici | gi189200538 |
| Aspergillus aculeatus | gi23267182 |

Putative EG II coding sequences have also been identified in Pseudotrichonympha grassii (gi15487326), Reticulitermes speratus (gi3800443), and Pyrenophora tritici-repentis (gi189201759, gi189200630, gi189200538).

TABLE 3
Exemplary EG III amino acid sequences

| Organism | Genbank Accession No. |
|---|---|
| Dimocarpus longan | gi254031741 |
| Trichoderma reesei | gi170549 |
| (Hypocrea jecorina) | |
| Trichoderma viride | gi33521680 |
| Aspergillus niger | gi145235569, gi134058120 |

EG polypeptides, and nucleic acids encoding them, from any of these and other organisms, can be used as described herein.

Variant EG cellulases can also be used in the present compositions and methods. Preferred variants retain the cellulase activity characteristic of naturally-occurring EG cellulases but may include amino acid mutations (i.e., substitutions, deletions, and/or insertions), or chemical modifications, that impart additional biochemical features. Such variants may be made using routine techniques in the field of recombinant genetics (See, e.g., Sambrook et al., Molecular Cloning, A Laboratory Manual, 2nd ed., 1989; Kriegler, Gene Transfer and Expression: A Laboratory Manual, 1990; and Ausubel et al., eds., CURRENT PROTOCOLS IN MOLECULAR BIOLOGY, Greene Publishing and Wiley-Interscience, New York, 1994). Common method for making amino acid mutations are site directed mutagenesis, PCR mutagenesis, and cassette mutagenesis.

The use of site-directed mutagenesis for making amino acid sequence variants of a starting polypeptide is well known in the art (see, e.g., Carter et al. Nucleic Acids Res. 13:4431-43 (1985) and Kunkel et al., Proc. Nat'l. Acad. Sci. U.S.A. 82:488 (1987)). Briefly, a starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of a starting gpolypeptide. See Hiuchi, in PCR Protocols, pp. 177-83 (Academic Press, 1990); and Vallette et al., Nuc. Acids Res. 17:723-33 (1989). See, also, e.g., Cadwell et al., PCR Methods and Applications, 2:28-33 (1992). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., Gene 34:315-23 (1985). The starting material is the plasmid (or other vector) comprising the starting polypeptide DNA to be mutated. The codon(s) in the starting DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a variant EG II can be determined, and a nucleic acid sequence encoding such amino acid sequence variant can be generated synthetically.

A variant polypeptide may include conservative amino acid substitutions that preserve the general charge, hydrophobicity/hydrophilicity, and/or steric bulk of the amino acid being substituted, while imparting other beneficial biochemical properties on the polypeptide. Non-limiting examples of conservative substitutions include those between the following groups: Gly/Ala, Val/Ile/Leu, Lys/Arg, Asn/Gln, Glu/Asp, Ser/Cys/Thr and Phe/Trp/Tyr. These and other conservative substitutions are shown in Table 4, below.

TABLE 4
Conservative amino acid replacements

| Original Amino Acid | Code | Conservative Substitution |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, |
| | | cis-3,4, or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Alternatively, the amino acid substitutions are not conservative and change the general charge, hydrophobicity/hydrophilicity, and/or steric bulk of the amino acid being substituted.

In some cases, an alignment of EG amino acid sequences is used to determine homology using a sequence comparison algorithm. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Nat'l. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection, Visual inspection may utilize graphics packages such as, for example, MOE by Chemical Computing Group, Montreal Canada.

An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (NCBI).

The BLAST algorithm allows the alignment of different EG amino acid sequences, different cellulase binding domains, or different cellulase enzymes, to identify amino acids that can likely be mutated to produce a variant having altered biochemical properties without significantly affecting the characteristic cellulase activity. These amino acids can be substituted as described, above, to produce a variant EG cellulase likely to possess cellulytic activity.

Also contemplated are chimeric EG polypeptides that include amino acid sequences a plurality of EG polypeptides, and consensus EG polypeptides determined by aligning a plurality of naturally-occurring EG amino acid sequences and selecting the predominant residues in each position for use in selecting an average or “consensus” amino acid sequence.

Variant polypeptides preferably retain the characteristic cellulolytic nature of the naturally-occurring polypeptide upon which they are based but have altered properties in some specific biochemical aspect. For example, a variant polypeptide may have a different pH or temperature optimum, altered temperature or oxidative stability, or altered sensitivity to salts, minerals, or other non-cellulosic components present in a slurry.

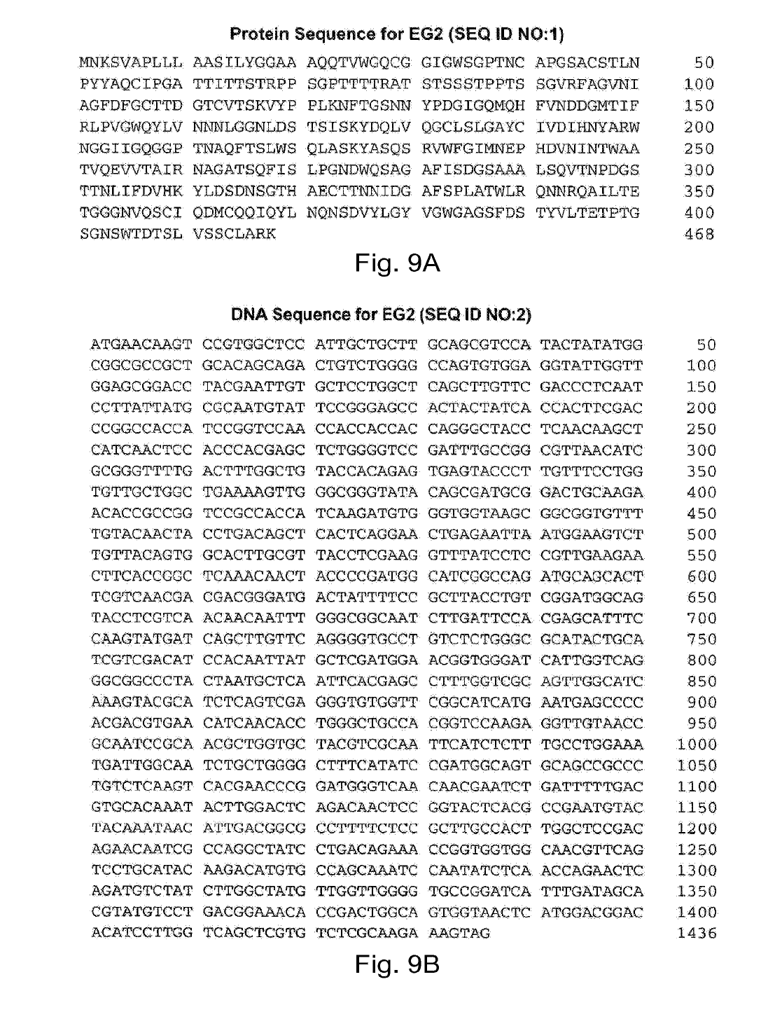

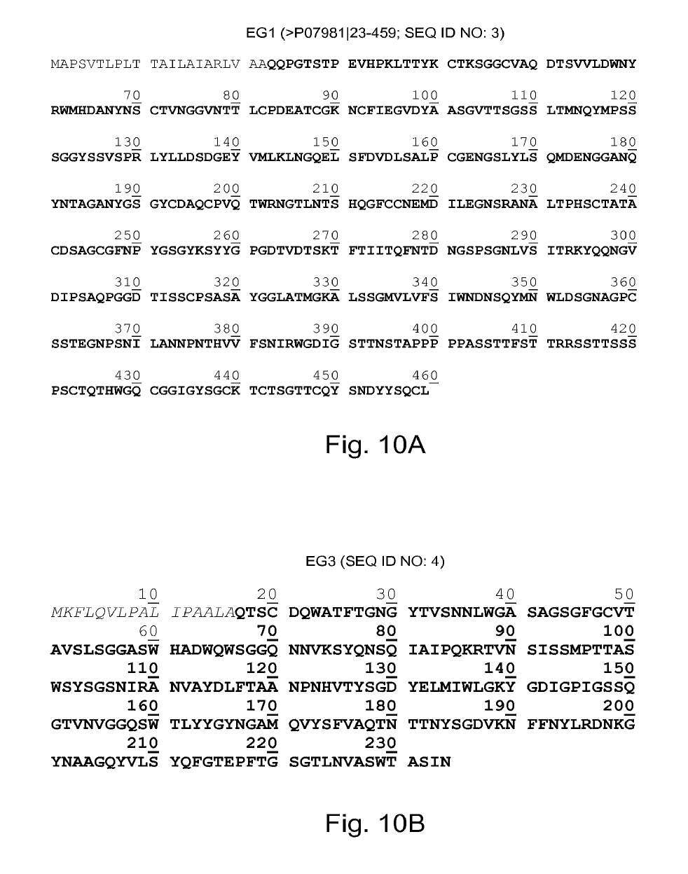

An exemplary variant EGII polypeptide is described in WO2008/088724 and shown as SEQ ID NO: 1 in FIG. 9A. The variant includes several amino acid substitutions with respect to the parent Trichoderma EGII polypeptide, e.g., V20A, E144D, and S400G. An exemplary EGI polypeptide is shown as SEQ ID NO: 3 in FIG. 10A. An exemplary EGIII polypeptide is shown as SEQ ID NO: 4 in FIG. 10B. The amino acid sequences of the mature polypeptides in FIGS. 10A and 10B are shown in bold. Additional EGIII polypeptides are described in, for example, U.S. Pat. Nos. 6,623,949, 6,635,465, 5,753,484, 7,094,588, 6,187,732, 6,268,328, 6,407,046, 6,500,211, 6,579,841, 6,582,750, 6,623,949, 6,635,465, and 7,501,272.

B. Cloning of EG Sequences

After a DNA sequence that encodes an EG cellulase or variant, thereof, has been identified or engineered, they may be inserted into a plasmid or vector (collectively referred to herein as “vectors”) by a variety of standard procedures. In general, the DNA sequence is inserted into an appropriate restriction endonuclease site(s) by standard procedures. Such procedures and related sub-cloning procedures are deemed to be within the scope of knowledge of those skilled in the art.

Heterologous nucleic acid constructs may include the coding sequence for an EG cellulase (i) in isolation; (ii) in combination with additional coding sequences; such as fusion protein or signal peptide coding sequences, where the EG-coding sequence is the dominant coding sequence; (iii) in combination with non-coding sequences, such as introns and control elements, such as promoter and terminator elements or 5′ and/or 3′ untranslated regions, effective for expression of the coding sequence in a suitable host; and/or (iv) in a vector or host environment in which the EG-coding sequence is a heterologous gene.

In one aspect of the present compositions and methods, a heterologous nucleic acid construct is employed to transfer an EG-encoding nucleic acid sequence into a cell in vitro, with established filamentous fungal and yeast lines preferred. For long-term, production of EG, stable expression is preferred. It follows that any method effective to generate stable transformants may be used in practicing the invention.

Appropriate vectors are typically equipped with a selectable marker-encoding nucleic acid sequence, insertion sites, and suitable control elements, such as promoter and termination sequences. The vector may comprise regulatory sequences, including, for example, non-coding sequences, such as introns and control elements, i.e., promoter and terminator elements or 5′ and/or 3′ untranslated regions, effective for expression of the coding sequence in host cells (and/or in a vector or host cell environment in which a modified soluble protein antigen coding sequence is not normally expressed), operably linked to the coding sequence. Large numbers of suitable vectors and promoters are known to those of skill in the art, many of which are commercially available and/or are described in Sambrook, et al., (supra).

Exemplary promoters include both constitutive promoters and inducible promoters, examples of which include a CMV promoter, an SV40 early promoter, an RSV promoter, an EF-1α promoter, a promoter containing the tet responsive element (TRE) in the tet-on or tet-off system as described (ClonTech and BASF), the β-actin promoter and the metallothionine promoter that can upregulated by addition of certain metal salts. A promoter sequence is a DNA sequence which is recognized by the particular filamentous fungus for expression purposes. It is operably linked to DNA sequence encoding an EG polypeptide. Such linkage comprises positioning of the promoter with respect to the initiation codon of the DNA sequence encoding the EG polypeptide in the disclosed expression vectors. The promoter sequence contains transcription and translation control sequence which mediate the expression of the EG polypeptide. Examples include the promoters from the Aspergillus niger, A awamori or A. oryzae glucoamylase, α-amylase, or α-glucosidase encoding genes; the A. nidulans gpdA or trpC Genes; the Neurospora crassa cbh1 or trp1 genes; the A. niger or Rhizomucor miehei aspartic proteinase encoding genes; the T. reesei (H. jecorina) cbh1, cbh2, egl1, egl2, or other cellulase encoding genes.

The choice of the proper selectable marker will depend on the host cell, and appropriate markers for different hosts are well known in the art. Typical selectable marker genes include argB from A. nidulans or T. reesei (H. jecorina), amdS from A. nidulans, pyr4 from Neurospora crassa or T. reesei, pyrG from Aspergillus niger or A. nidulans. Additional exemplary selectable markers include, but are not limited to trpc, trp1, oliC31, niaD, or leu2, which are included in heterologous nucleic acid constructs used to transform a mutant strain such as trp⁻, pyr⁻, leu⁻ and the like.

Such selectable markers confer to transformants the ability to utilize a metabolite that is usually not metabolized by the filamentous fungi. For example, the amdS gene from T. reesei (H. jecorina) which encodes the enzyme acetamidase that allows transformant cells to grow on acetamide as a nitrogen source. The selectable marker (e.g., pyrG) may restore the ability of an auxotrophic mutant strain to grow on a selective minimal medium or the selectable marker (e.g., olic31) may confer to transformants the ability to grow in the presence of an inhibitory drug or antibiotic.

The selectable marker coding sequence is cloned into any suitable plasmid using methods generally employed in the art. Exemplary plasmids include pUC18, pBR322, pRAX and pUC100. The pRAX plasmid contains AMAL sequences from A. nidulans, which make it possible to replicate in A. niger.

Any vector may be used as long as it is replicable and viable in the cells into which it is introduced. Large numbers of suitable vectors and promoters are known to those of skill in the art, and are commercially available. Cloning and expression vectors are also described in Sambrook et al., 1989, Ausubel, F. M. et al., 1989, and Strathern et al., The Molecular Biology of the Yeast Saccharomyces, 1981, each of which is expressly incorporated by reference herein. Appropriate expression vectors for fungi are described in van den Hondel, C. A. M. J. J. et al. (1991) In: Bennett, J. W. and Lasure, L. L. (eds.) More Gene Manipulations in Fungi. Academic Press, pp. 396-428.

C. Expression of EG

As described above, EG is naturally expressed in many filamentous fungi, including Trichoderma reesei (Hypocrea jecorina), Trichoderma viride, Penicillium janthinellum, Volvariella volvacea, and Sclerotinia sclerotiorum, with EG coding sequence being identified in several others. In some cases, EG can be isolated from such natural sources without the need to clone and express the enzyme in a heterologous host organism. However, in many cases it is desirable to over-express EG in a host organism capable of producing more of the polypeptide than is typically expressed in naturally-occurring organisms.

In some cases, it may advantageous to express EG in a transformation host that bears phylogenetic similarity to the source organism for the EG, particularly where codon usage or post-translational processing are a concern. In other cases, it may advantageous to express the EG in a different host cell that is not phylogenetic related. The skilled person will be capable of selecting the best expression system for a particular gene through routine techniques utilizing the tools available in the art.

In one example, the microorganism to be transformed for the purpose of expressing an EG polypeptide is a strain derived from Trichoderma sp. Thus, a preferred mode for preparing EG for use as described involves transforming a Trichoderma sp. host cell with a DNA construct comprising at least a fragment of DNA encoding a portion or all of the EG. A suitable DNA construct will generally be functionally attached to a promoter, optionally with other transcription or translation regulatory sequences to optimize expression. A transformed host cell is then grown under conditions so as to express the desired protein. Subsequently, the desired protein product may be purified to substantial homogeneity. In an alternative embodiment, Aspergillus niger can be used as an expression vehicle. For a description of transformation techniques with A. niger, see WO 98/31821, the disclosure of which is incorporated by reference in its entirety.

EG is preferably secreted from the cells to avoid the need separate the polypeptide from cell proteins. However, EG can also be expressed as an intracellular protein. The culture conditions, such as temperature, pH and the like, are generally those known to be use with the particular host cells and will be apparent to the skilled person.

Exemplary expression systems are described in more detail, below.

1. Filamentous Fungi

Examples of species of filamentous fungi that may be used for EG expression include, but are not limited to Trichoderma, e.g., Trichoderma reesei, Trichoderma longibrachiatum, Trichoderma viride, Trichoderma koningii; Penicillium sp., Humicola sp., including Humicola insolens; Aspergillus sp., Chrysosporium sp., Fusarium sp., Hypocrea sp., and Emericella sp.

EG expressing cells are cultured under conditions typically employed to culture the parental fungal line. Generally, cells are cultured in a standard medium containing physiological salts and nutrients, such as described in Pourquie, J. et al., Biochemistry and Genetics of Cellulose Degradation, eds. Aubert, J. P. et al., Academic Press, pp. 71-86, 1988 and Ilmen, M. et al., Appl. Environ. Microbiol. 63:1298-1306, 1997. Culture conditions are also standard, e.g., cultures are incubated at 28° C. in shaker cultures or fermenters until desired levels of EG expression are achieved.

Preferred culture conditions for a given filamentous fungus may be found in the scientific literature and/or from the source of the fungi such as the American Type Culture Collection (ATCC). After fungal growth has been established, the cells are exposed to conditions effective to cause or permit the expression of EG. In cases where an EG coding sequence is under the control of an inducible promoter, the inducing agent, e.g., a sugar, metal salt or antibiotics, is added to the medium at a concentration effective to induce EG expression.

In one embodiment, the strain comprises Aspergillus niger, which is a useful strain for obtaining overexpressed protein. For example A. niger var awamori dgr246 is known to secrete elevated amounts of secreted cellulases (Goedegebuur et al., Curr. Genet., 41: 89-98, 2002). Other strains of A. niger var awamori such as GCDAP3, GCDAP4 and GAP3-4 are known (Ward, M. et al., Appl. Microbiol. Biotechnol. 39:738-43, 1993).

In another embodiment, the strain comprises Trichoderma reesei, which is a useful strain for obtaining overexpressed protein. For example, RL-P37, described by Sheir-Neiss, et al., Appl. Microbiol. Biotechnol. 20:46-53, 1984) is known to secrete elevated amounts of cellulase enzymes. Functional equivalents of RL-P37 include Trichoderma reesei strain RUT-C30 (ATCC No. 56765) and strain QM9414 (ATCC No. 26921). It is contemplated that these strains would also be useful in over-expressing EG.

Where it is desired to obtain the EG in the absence of potentially detrimental native cellulolytic activity, it is useful to obtain a Trichoderma host cell strain which has had one or more cellulase genes deleted prior to introduction of a DNA construct or plasmid containing the DNA fragment encoding EG. Such strains may be prepared by the method disclosed in U.S. Pat. No. 5,246,853 and WO 92/06209, which disclosures are hereby incorporated by reference. By expressing a EG cellulase in a host microorganism that is missing one or more cellulase genes, the identification and subsequent purification procedures are simplified. Generally, any gene from Trichoderma sp. which has been cloned can also be deleted, for example, the cbh1, cbh2, egl1, and egl2 genes as well as those encoding EG III and/or EG V protein (see, e.g., U.S. Pat. No. 5,475,101 and WO 94/28117, respectively).

Gene deletion may be accomplished by inserting a form of the desired gene to be deleted or disrupted into a plasmid by methods known in the art. The deletion plasmid is then cut at an appropriate restriction enzyme site(s), internal to the desired gene coding region, and the gene coding sequence or part thereof replaced with a selectable marker. Flanking DNA sequences from the locus of the gene to be deleted or disrupted, preferably between about 0.5 to 2.0 kb, remain on either side of the selectable marker gene. An appropriate deletion plasmid will generally have unique restriction enzyme sites present therein to enable the fragment containing the deleted gene, including flanking DNA sequences, and the selectable marker gene to be removed as a single linear piece.

A selectable marker may be chosen to enable detection of the transformed microorganism. Any selectable marker gene that is expressed in the selected microorganism will be suitable. For example, with Aspergillus sp., the selectable marker is chosen so that the presence of the selectable marker in the transformants will not significantly affect the properties thereof. Such a selectable marker may be a gene that encodes an assayable product. For example, a functional copy of an Aspergillus sp. gene may be used which if lacking in the host strain results in the host strain displaying an auxotrophic phenotype. Similarly, selectable markers exist for Trichoderma sp.

In one embodiment, a pyrG-derivative strain of Aspergillus sp. is transformed with a functional pyrG gene, which thus provides a selectable marker for transformation. A pyrG-derivative strain may be obtained by selection of Aspergillus sp. strains that are resistant to fluoroorotic acid (FOA). The pyrG gene encodes orotidine-5′-monophosphate decarboxylase, an enzyme required for the biosynthesis of uridine. Strains with an intact pyrG gene grow in a medium lacking uridine but are sensitive to fluoroorotic acid. It is possible to select pyrG-derivative strains that lack a functional orotidine monophosphate decarboxylase enzyme and require uridine for growth by selecting for FOA resistance. Using the FOA selection technique it is also possible to obtain uridine-requiring strains which lack a functional orotate pyrophosphoribosyl transferase. It is possible to transform these cells with a functional copy of the gene encoding this enzyme (Berges and Barreau, Curr. Genet. 19:359-65, 1991), and van Hartingsveldt et al., Mol. Gen. Genet. 206:71-75, 1986). Selection of derivative strains is easily performed using the FOA resistance technique referred to above, and thus, the pyrG gene is preferably employed as a selectable marker.

In a second embodiment, a pyr4-derivative strain of Trichoderma sp. is transformed with a functional pyr4⁻ gene, which thus provides a selectable marker for transformation. A pyr4⁻ derivative strain may be obtained by selection of Trichoderma sp. strains that are resistant to fluoroorotic acid (FOA). The pyr4 gene encodes orotidine-5′-monophosphate decarboxylase, an enzyme required for the biosynthesis of uridine. Strains with an intact pyr4 gene grow in a medium lacking uridine but are sensitive to fluoroorotic acid. It is possible to select pyr4⁻ derivative strains that lack a functional orotidine monophosphate decarboxylase enzyme and require uridine for growth by selecting for FOA resistance. Using the FOA selection technique it is also possible to obtain uridine-requiring strains which lack a functional orotate pyrophosphoribosyl transferase. It is possible to transform these cells with a functional copy of the gene encoding this enzyme (Berges and Barreau, 1991, supra). Selection of derivative strains is easily performed using the FOA resistance technique referred to above, and thus, the pyr4 gene is preferably employed as a selectable marker.

To transform pyrG⁻ Aspergillus sp. or pyr4⁻ Trichoderma sp. so as to be lacking in the ability to express one or more cellulase genes, a single DNA fragment comprising a disrupted or deleted cellulase gene is then isolated from the deletion plasmid and used to transform an appropriate pyr⁻ Aspergillus or pyr⁻ Trichoderma host. Transformants are then identified and selected based on their ability to express the pyrG or pyr4, respectively, gene product and thus compliment the uridine auxotrophy of the host strain. Southern blot analysis is then carried out on the resultant transformants to identify and confirm a double crossover integration event that replaces part, or all, of the coding region of the genomic copy of the gene to be deleted with the appropriate pyr selectable markers.

Although the specific plasmid vectors described above relate to preparation of pyr transformants, the present invention is not limited to these vectors. Various genes can be deleted and replaced in the Aspergillus sp. or Trichoderma sp. strain using the above techniques. In addition, any available selectable markers can be used, as discussed above. In fact, any host, e.g., Aspergillus sp. or Trichoderma sp., gene that has been cloned, and thus identified, can be deleted from the genome using the above-described strategy.

As stated above, the host strains used may be derivatives of Trichoderma sp. that lack or have a nonfunctional gene or genes corresponding to the selectable marker chosen. For example, if the selectable marker of pyrG is chosen for Aspergillus sp., then a specific pyrG⁻ derivative strain is used as a recipient in the transformation procedure. Also, for example, if the selectable marker of pyr4 is chosen for a Trichoderma sp., then a specific pyr4⁻ derivative strain is used as a recipient in the transformation procedure. Similarly, selectable markers comprising Trichoderma sp. genes equivalent to the Aspergillus nidulans genes amdS, argB, trpC, niaD may be used. The corresponding recipient strain must therefore be a derivative strain such as argB-, trpC-, niaD-, respectively.

DNA encoding EG II and appropriate regulatory sequences is then prepared for insertion into an appropriate microorganism, and may be functionally attached to a fungal promoter sequence, for example, the promoter of the glaA gene in Aspergillus or the promoter of the cbh1 or egl1 genes in Trichoderma.

The expression vector used to carry DNA encoding EG may be any vector which is capable of replicating autonomously in a given host organism or of integrating into the DNA of the host, typically a plasmid. Two general types of expression vectors for obtaining expression of EG are contemplated. The first contains DNA sequences in which the promoter, gene-coding region, and terminator sequence all originate from the gene to be expressed. The second type of expression vector is preassembled and contains sequences required for high-level transcription and a selectable marker. It is contemplated that the coding region for a gene or part thereof can be inserted into this general-purpose expression vector such that it is under the transcriptional control of the expression cassettes promoter and terminator sequences. An exemplary general-purpose expression vector for use in Aspergillus is pRAX, in which the EG coding sequence is inserted downstream of the strong glaa promoter. A general-purpose expression vector for expression in Trichoderma is pTEX, in which the EG coding sequence can be inserted downstream of the strong cbh1 promoter. More generally, the promoter may be any DNA sequence that shows transcriptional activity in the host cell and may be derived from genes encoding proteins either homologous or heterologous to the host cell. An optional signal peptide provides for extracellular production of EG. The DNA encoding the signal sequence may be that which is naturally associated with the gene to be expressed; however, the signal sequence from any suitable source, for example an exo-cellobiohydrolase or endoglucanase from Trichoderma can also be used.

The DNA vector or construct described above may be introduced in the host cell in accordance with known techniques such as transformation, transfection, microinjection, microporation, biolistic bombardment and the like.

The permeability of the cell wall of fungal cells such as Trichoderma sp. and Aspergillus sp to DNA is very low; therefore, the uptake of a DNA sequence, gene, or gene fragment is at best minimal. However, there are a number of methods to increase the permeability of the cell wall. The preferred method involves the preparation of protoplasts from fungal mycelium (see, e.g., Campbell et al. Curr. Genet. 16:53-56, 1989.) In this method, the mycelium may be obtained from germinated vegetative spores and treated with an enzyme that digests the cell wall to produce protoplasts. The protoplasts are then protected by osmotic stabilizer in the suspending medium. These stabilizers include sorbitol, mannitol, potassium chloride, magnesium sulfate and the like, usually at a concentration of from 0.8 M to 1.2 M. Where sorbital is used, it is preferable to use an about 1.2 M solution.

Uptake of the DNA into protoplasts is dependent upon the calcium ion concentration, which is generally between about 10 mM CaCl₂ and 50 mM. The calcium ion uptake solution also generally includes a buffering system such as TE buffer (10 mM Tris, pH 7.4; 1 mM EDTA) or 10 mM MOPS (morpholinepropanesulfonic acid), pH 6.0 buffer, and polyethylene glycol (PEG).

Aspergillus sp. protoplast are usually transformed at a density of 10⁵ to 10⁶ per mL, and preferably 2×10⁵ per mL, while Trichoderma sp. protoplasts are usually transformed at a density of 10⁸ to 10⁹ per mL, and preferably 2×10⁸ per mL. Routinely, a volume of about 100 μL of protoplasts in an appropriate solution (e.g., 1.2 M sorbitol; 50 mM CaCl₂) is mixed with the desired DNA and from 0.1 to 1 volume of 25% PEG 4000 is added to the protoplast-DNA suspension. Additives such as dimethyl sulfoxide, heparin, spermidine, potassium chloride, and the like, may also be added to the uptake solution to enhance transformation.

The protoplast-DNA mixture is then incubated at approximately 0° C. for a period of between 10 to 30 minutes. Additional PEG may be added to further enhance the uptake of DNA. The transformation mixture is then incubated either at room temperature or on ice before the addition of a sorbitol and CaCl₂ solution. The protoplast suspension is then added to molten aliquots of a selective growth medium that only permits the growth of transformants. For example, if pyr⁺ transformants are being selected for, it is preferable to use a growth medium that lacks uridine. Subsequent colonies are transferred and grown in growth medium lacking uridine.

Stable transformants may be distinguished from unstable transformants by their faster growth rate. In Trichoderma, for example, the formation of circular colonies with a smooth, rather than ragged, outline on solid culture medium lacking uridine is indicative of stable transformation. Additionally, in some cases a further test of stability may made by growing the transformants on solid non-selective medium (e.g., containing uridine), harvesting spores, and determining the percentage of these spores that subsequently germinate and grow on selective medium lacking uridine.

2. Yeast

Yeast (i.e., non-filamentous fungi) may also be used as a host cell for EG production. Other genes encoding hydrolytic enzymes have been expressed in various strains of the yeast S. cerevisiae, including two endoglucanases (Penttila et al., Yeast 3:175-185, 1987), two cellobiohydrolases (Penttila et al., Gene, 63:103-112, 1988), and one β-glucosidase from Trichoderma reesei (Cummings and Fowler, Curr. Genet. 29:227-33, 1996), a xylanase from Aureobasidlium pullulans (Li and Ljungdahl, Appl. Environ. Microbiol. 62:209-13, 1996), an alpha-amylase from wheat (Rothstein et al., Gene 55:353-56, 1987), etc. In addition, a cellulase gene cassette encoding the Butyrivibrio fibrisolvens endo-β-1,4-glucanase (END1), Phanerochaete chrysosporium cellobiohydrolase (CBH 1), the Ruminococcus flavefaciens cellodextrinase (CEL1), and the Endomyces fibrilizer cellobiase (Bgl1) was successfully expressed in a laboratory strain of S. cerevisiae (Van Rensburg et al., Yeast, 14:67-76, 1998).

Yeast can generally be transformed by preparing protoplasts, as described above. Yeast transformation is well-known in the art and not described in detail, herein.

D. Isolation and Purification of EG

In some embodiments of the compositions and methods, EG is substantially isolated from other cellular or media components, including other cellulases, present in or secreted by a host organism. In this manner, the compositions are distinct from crude cellular extract or crude media that contain EG as one of a plurality of cellulases. In the present compositions and methods, EG preferably represents at least 70%, at least 80%, preferably at least 90%, at least 91%, at least 92%, at least 93%, at least 94%, at least 95%, at least 96%, at least 97%, at least 98%, or even at least 99% (wt/wt) of the total protein present in a composition. In particular embodiments, the present EG compositions are substantially free of CBH-type cellulases, BG-type cellulases, and/or xylanases.

In some cases, EG may be produced in cell culture and secreted into the medium, thereby avoiding the need to isolate EG from cellular proteins. In other cases, EG may be produced in a cellular form, necessitating recovery from a cell lysate. In either case, techniques for isolating polypeptides are well-known in the art and include but are not limited to, affinity chromatography (Tilbeurgh et al., FEBS Lett. 16:215, 1984); ion-exchange chromatographic methods (Goyal et al., Bioresource Technol. 36:37-50, 1991; Fliess et al., Eur. J. Appl. Microbiol. Biotechnol. 17:314-18, 1983; Bhikhabhai et al., J. Appl. Biochem. 6:336-45, 1984; Ellouz et al., J. Chromatography 396:307-17, 1987), including ion-exchange using materials with high resolution power (Medve et al., J. Chromatography A 808:153-65, 1998); hydrophobic interaction chromatography (Tomaz and Queiroz, J. Chromatography A 865:123-28, 1999); gel filtration/molecular exclusion chromatography; reverse phase HPLC; antibody-affinity column chromatography; metal chelate chromatography; ammonium sulfate precipitation; two-phase partitioning (Brumbauer, et al., Bioseparation 7:287-95, 1999); ethanol precipitation; chromatofocusing; isoelectric focusing; SDS-PAGE; and the like. Various methods of protein purification may be employed and such methods are known in the art and described e.g., in Deutscher, Methods in Enzymology, 182:779, 1990; Scopes, Methods Enzymol. 90:479-91, 1982. The purification step(s) selected will depend, e.g., on the nature of the production process and the desired level of purity.

As detailed, herein. EG may be produced in a host organism in which other cellulase genes have been deleted. Particularly, when a gene encoding EG is placed under the control or an efficient promoter, EG can be expressed and secreted at such a high level that it represents the vast majority of the protein present in the media, making further purification unnecessary.

### VI. Utility of EG Compositions and Methods

The present compositions and methods find use in reducing the viscosity of plant material slurries. Such slurries include grain mash, wort, fermentation broth, and other liquid plant material mixtures, suspensions, solutions, and combinations, thereof. Plant material slurries are frequently viscous due to the presence of cellulose materials that must be hydrolyzed to facilitate mixing, transfer (including gravity feed and pumping), filtration, and other manipulations of the slurry.

Conventional compositions and methods for reducing the viscosity of plant material slurries involve the use of a mixture of cellulases, which were typically only partially-defined. An exemplary mixture of cellulases is found in the product OPTIMASH™ BG (Danisco, Genencor Division, Palo Alto, Calif.), a product that contains crude filamentous fungi (Trichoderma sp.) culture media into which various cellulases have been secreted. OPTIMASH™ BG contains about 50-60% EG II along with significant amounts of EG I, β-glucanase, and β-glucosidase activity, as well as laminarase and hemicellulase activities, indicating the presence of a variety of different cellulytic and hemicellultic enzymes. Because OPTIMASH™ BG is a whole cell broth-type product, it also contains other secreted proteins, and media components. While the use of cellulase mixtures to reducing slurry viscosity can produce acceptable results for some applications, the introduction of a mixture of cellulases and other proteins increases the possibility that one or more of the component will interfere with subsequent processing steps, including fermentation. Moreover, crude culture medium may be subject to batch-to-batch variability, particularly where the relative amounts of different cellulases and other components are not well defined. While it would be possible to isolate each cellulase in a crude culture medium and reconstitute purified cellulases into a product, the additional steps would greatly increase the cost of production, and, therefore, the cost to an end user.

The present compositions and methods are based on the unexpected observation that an EG-type cellulase alone, in the absence of other types of cellulases, is sufficient to reduce the viscosity of certain plant material slurries. This discovery allows a single cellulase, isolated and purified to a desired level, to be used as a viscosity-reducing agent, avoiding the introduction of addition cellulases and other unnecessary components into a slurry. A composition comprising a single cellulase can be subjected to any level of quality control, activity assessment, and other validation selected by the manufacturer and end user.

A cellulase composition containing only EG, or even a single EG, is particularly useful for reducing the viscosity of plant material slurries in which betaglucan, rather than pensosans, is primarily responsible for the viscosity of the slurry. For example, data obtained in support of the compositions and methods demonstrated that EG was at least as effective as a mixed cellulase composition (i.e., OPTIMASH™ BG) in reducing the viscosity of a barley slurry, which contained about 3-5% betaglucan and 14.4% non-cellulosic polysaccharides, but less effective than the mixed cellulase composition in reducing the viscosity of a wheat slurry, which contained about 1% betaglucan and 9.9% non-cellulosic polysaccharides (Bach Knudsen K. E., Animal Feed Science Technology 67: 319-38, 1997; Englyst et al., J. Sci. Food Agric. 34: 1434-40, 1983).

Oat and barley slurries include similar amounts of betaglucan and pentosans; therefore, the present EG compositions are likely to reduce the viscosity of oat slurries, in addition to barley slurries. More generally, the EG compositions are likely to reduce the viscosity of any plant material slurry in which betaglucan is primarily responsible for viscosity. In particular cases, such slurries contain at least about as much betaglucan as pentosans by weight. A discussion of the relative amount of betaglucan and pentosans in different grains can be found, e.g., in Genç, H. et al., Food Chemistry 73:221-24, 2001; Henry, R. J., J. Sci. Food and Agriculture, 36:1243-53, 1985; Henry, R. J., J. Cereal Sci. 6:253-58, 1987; Brunner, B. R. and Freed, R. D., Crop Sci. 34:473-76, 1994; Welch, R. W. and Lloyd, J. D., Cereal Sci. 9:35-40, 1989; and Peterson, D. M., Crop Sci. 31:1517-20, 1991.

The amount of EG composition that must be added to a slurry to reduce its viscosity varies depending on the concentration of the slurry, the desired reduction in viscosity, the time allotted for the process, pH, temperature, and the like. As shown in the Examples Example 3, an amount of about 0.0164 kg EG composition per metric ton of barley material was sufficient to reduce the viscosity of the slurry below that obtained using the mixed cellulase composition. Thus, one can readily envision the use of from 0.01-0.5 kg EG per metric ton, or even 0.005-1 kg EG per metric ton slurry.

The present EG compositions and methods can also be used to reduce slurry viscosity at a higher temperature than is possible using a mixed cellulase composition. For example, as shown in Example 4, the EG II composition was effective at temperatures up to about 75° C., while the mixed the cellulase composition was effective only up to about 65° C. Thus, the EG II compositions and methods enable viscosity reduction at temperatures greater than about 65° C., such as 65° C.-75° C.

It will be appreciated from the foregoing that the present compositions and methods find utility in a wide variety applications, including brewing and whiskey production, animal feed and health food production, and ethanol production. Other embodiments and uses of the present compositions will be apparent to the skilled person from foregoing description and following examples.

### EXAMPLES

The present compositions and methods are illustrated by the following Examples which are in no way intended to be limiting.

Unless otherwise indicated, the following abbreviations are used: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); V (volts); MW (molecular weight); sec (seconds); min(s) (minute/minutes); h(s) and hr(s) (hour/hours); ° C. (degrees Centigrade); QS (quantity sufficient); ND (not done); NA (not applicable); rpm (revolutions per minute); H₂O (water); dH₂O (deionized water); HCl (hydrochloric acid); aa (amino acid); by (base pair); kb (kilobase pair); kD (kilodaltons); cDNA (copy or complementary DNA); DNA (deoxyribonucleic acid); ssDNA (single stranded DNA); dsDNA (double stranded DNA); dNTP (deoxyribonucleotide triphosphate); RNA (ribonucleic acid); MgCl₂ (magnesium chloride); NaCl (sodium chloride); w/v (weight to volume); v/v (volume to volume); g (gravity); OD (optical density); CNPG (chloro-nitro-phenyl-beta-D-glucoside); CNP (2-chloro-4-nitrophenol); APB (acid-pretreated bagasse); PAGE (polyacrylamide gel electrophoresis); PCR (polymerase chain reaction); RT-PCR (reverse transcription PCR); and HPLC (high pressure liquid chromatography). Terms and abbreviation that are not expressly defined should be afforded their ordinary meaning as used in the relevant art.

### EXAMPLES

The following examples are provided to illustrate the present compositions and methods and should not be construed as limiting.

### Example 1

### EG II Expression

Construction of host cells to express of a modified EGII cellulase was performed substantially as described in WO2008/088724, using a strain of T. reesei in which the major cellulases (i.e., CBH I, CBH II, EG I, and EG II) were deleted or disrupted (see, e.g., U.S. Pat. No. 5,472,864 and WO 92/17574 for techniques relating to the deletion of cellulase genes. The host cells were transformed with a single copy of an expression vector derived from pTrex3 (see U.S. Pat. No. 6,426,410), which containing the nucleotide sequence of a variant Trichoderma EG II gene (SEQ ID NO: 2 in FIG. 9B, described in WO2008/088724) under the control of the CBH I promoter. The corresponding amino acid sequence is shown as SEQ ID NO: 1 in FIG. 9A.

### Example 2

### Biochemical Characterization of EG II Enriched T. Reesei Products

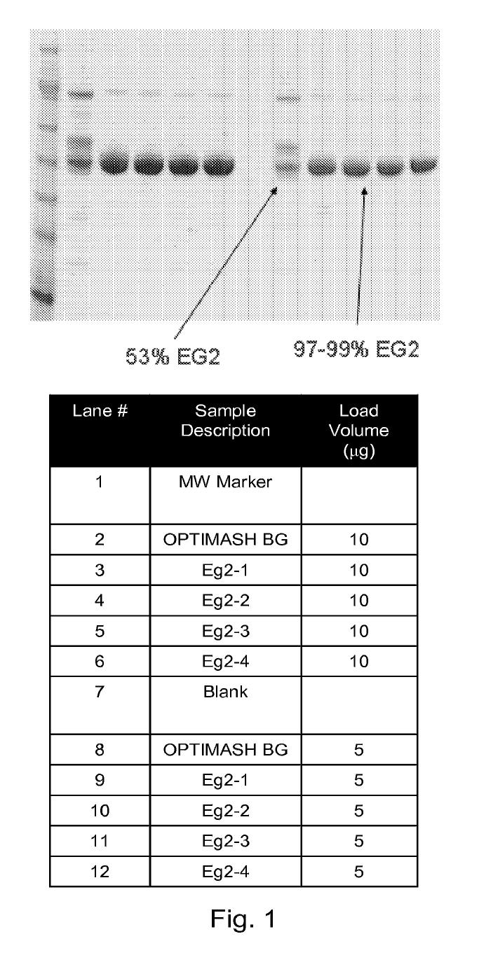

EG II was recovered and characterized as described in WO2008/088724. Briefly, the Morph 1.1 strain of T. reesei producing EG II under control of the CBH 1 promoter (see Example 1) was grown in a fermentor using standard methods. The supernatant was recovered and concentrated. SDS-PAGE analysis followed by densitometry scanning indicated that the supernatant contained about 93-99% (i.e., about 96%) EG II protein (FIG. 1, lanes 3-6 and 9-12).

OPTIMASH™ BG is a commercially-available (Genencor International, Palo Alto, Calif., USA), mixed-cellulase product used to reduce the viscosity of plant material compositions. SDS-PAGE analysis followed by densitometry scanning indicated that the major components in the supernatant were about 53% EG II protein and 35% EG 1 protein (FIG. 1, lanes 2 and 8). A noted, above, OPTIMASH™ BG contains a variety of other cellulytic and hemicellulytic activities.

The EG II supernatant and OPTIMASH™ BG were concentrated and formulated with either 13% sorbitol, 1.35% sodium benzoate, or 13% glycerol, such that they contained the same amount of EG II protein. Protein concentration was based on scatter-corrected A 280 nm measurements, which measure the intrinsic absorption of proteins due to the presence of aromatic amino acids in their composition (mainly tyrosine and tryptophan). More accurate measurements are obtained if with the absorbance is corrected for scattering due to the possible presence of interfering substances in the sample. Using these measurement, EG II was concentrated to 78 g/L protein and OPTIMASH™ BG was formulated to 124 g/L protein (scatter-corrected A 280 nm).

The results shown in FIG. 1 indicate that EG II protein is the predominant protein present in supernatant obtained from the Morph 1.1 strain of T. reesei transformed with a gene encoding a EG II protein. Such supernatant can be used directly and without further purification as a source of EG II protein.

### Example 3

### Viscosity Reduction Using EG II Compositions

European wheat or barley (van Beelen diervoeders) was used for these experiments. The wheat or barley material was milled at 10,000 rpm/2 mm sieve, resulting in a size distribution in which 86.2% of the particles were <1.0 mm nominal diameter. Slurries containing 25-28% dissolved solids were prepared in a mixture of 50/50 demi water/tap water. The initial pH of this slurry was about pH 6, which was adjusted to pH 3.7 using 4 N H₂SO₄. 100 grams of slurry was used per viscosity measurement.

### Viscosity Measurements

Viscosities were measured with a Haake Viscotester 550 using a standardized protocol. An external water bath (DC30) was used for temperature regulation along with an FL10 sensor system with a star shaped rotor (γ=50 1/s=>Ω=58.58 1/min) Enzymes were added at the beginning of the viscosity measurements. Viscosity profiles were followed for 90 minutes at 56-57° C. (excluding warming up time). After this period of time, the slurries were cooled to 32° C., and the viscosity was measured at fermentation temperature.

In a variation of the method, viscosities were measured using a Brookfield DV-E viscometer, at a setting of 50 rpm, along with a S63 spindle. Viscosity was monitored after 30 minutes, or the average of viscosities measured at 25, 30, and 35 minutes were used.

### Viscosity Reduction in Wheat

An experiment was performed to determine whether a composition substantially consisting of EG II could be used to reduce the viscosity of a wheat composition, which contains approximately 1% betaglucan and 6-8% pentosans. In this experiment, EG II was compared to the commercially available product OPTIMASH™ BG in a process that mimicked a no-cook process.

Typically, a dosage of about 0.03-0.06 kilogram per metric ton (kg/MT) OPTIMASH™ BG is needed for efficient viscosity reduction in a wheat composition using a standard viscosity reduction process (i.e., 30 min at 60° C.; 2 hr at 85° C.). In a no-cook process (i.e., activation step 60-90 min at 55° C.; fermentation 48-50 hr at 30° C.) a dosage of about 0.10 kg/MT OPTIMASH™ BG is needed.

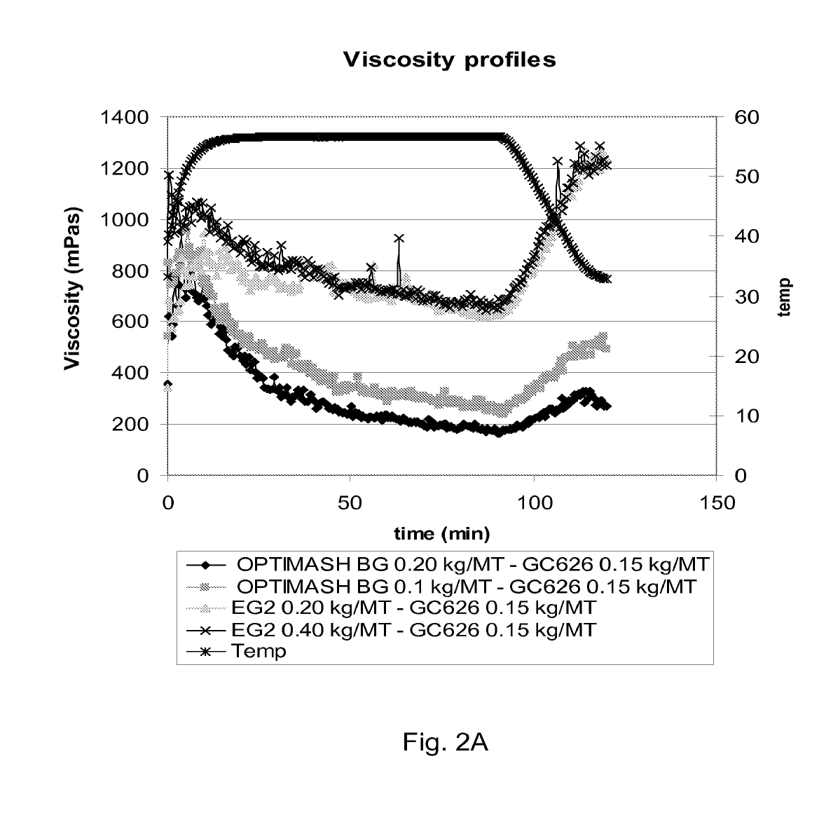

The results of viscosity measurements obtained by incubating different amounts of either EG II or OPTIMASH™ BG with 30% dissolved solid wheat (van Beelen #200706, milling 2 mm sieve, 10,000 rpm) for 60 min at 55° C. and pH 3.6 are shown in FIG. 2A. These conditions mimic a “no-cook” or “low temperature” liquefaction process.

The results of viscosity measurements obtained by incubating 0.4 kg/MT EG II or 0.1 kg/MT OPTIMASH™ BG with 30% dissolved solid wheat (van Beelen #200706, milling 2 mm sieve, 10,000 rpm) for 30 min 60 C and 2 hr at 85° C. and pH 5.5 are shown in FIG. 2B. These conditions mimic a conventional liquefaction process.

In both cases, OPTIMASH™ BG reduced the viscosity of the wheat composition more than EG II, suggesting that EG II, alone, is not effective in reducing the viscosity of a wheat composition.

### Viscosity Reduction in Barley

An experiment was performed to determine whether a composition substantially consisting of EG II could be used to reduce the viscosity of a barley (van Beelen #2007116) composition, which contains approximately 3-5% betaglucan and 2-4% pentosans. As above, EG II was compared to the commercially available product OPTIMASH™ BG. Typically, a dosage of about 0.05 kilogram per metric ton (kg/MT) OPTIMASH™ BG is needed for efficient viscosity reduction in a barley composition using a standard viscosity reduction process (i.e., 30 min at 60° C.; 2 hr at 85° C.). In a no-cook STARGEN process (i.e., activation step 60-90 min at 55° C.; fermentation 48-50 hr at 30° C.) a dosage of about 0.20 kg/MT OPTIMASH™ BG is needed.

A first experiment was performed to mimic a conventional liquefaction process, wherein a barley composition was preheated for 30 min. at 60° C., then subjected to a liquefaction step for 2 hrs at 85° C. Equivalent protein doses of no enzyme (host strain), OPTIMASH™ BG, or EG II were added prior to the preheating step.

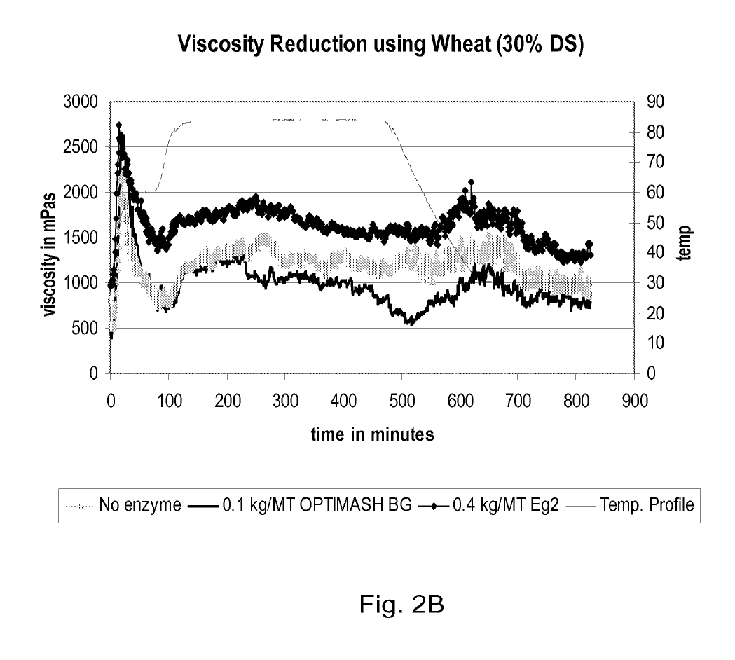

As shown in FIG. 3, EG II was as effective as OPTIMASH™ BG in reducing the viscosity of the barley slurry, whereas the no enzyme control, containing the proteins of the host strain used for the expression of EG II, did not reduce the viscosity of the barley slurry.

A second experimental setup mimicked a conventional jet cooker process, wherein a barley composition is heated in a jet cooker for 10 min at 120-130° C., subjected to a first liquefaction for 65 min at 83° C., subjected to a second liquefaction for 65 min at 83° C., and then subjected to a third liquefaction for 20-30 min at 70° C. The protocol in Table 5 was used to evaluate the reduction of viscosity of a barley composition.

TABLE 5
Jet cooker process for starch liquefaction

| Step | Conditions |
|---|---|
| Slurry make-up | 20 minutes at 55° C. |
| Boiling (to mimic jet-cooker step) | 10 minutes at 100° C. |
| Primary liquefaction: | 65 minutes at 83° C. |
| Secondary liquefaction: | 65 minutes at 83° C. |
| Third liquefaction: | 20-30 minutes at 70° C. |

The barley slurry had 32% dissolved solids and a pH of 5.7-5.8. Equivalent doses (kg enzyme/MT slurry) EG II, OPTIMASH™ BG, or no enzyme (as a control) was added in the slurry make-up (A S/M). Where indicated, additional EG II, OPTIMASH™ BG, or no enzyme (as a control) was added before the third liquefaction (A Boiling).

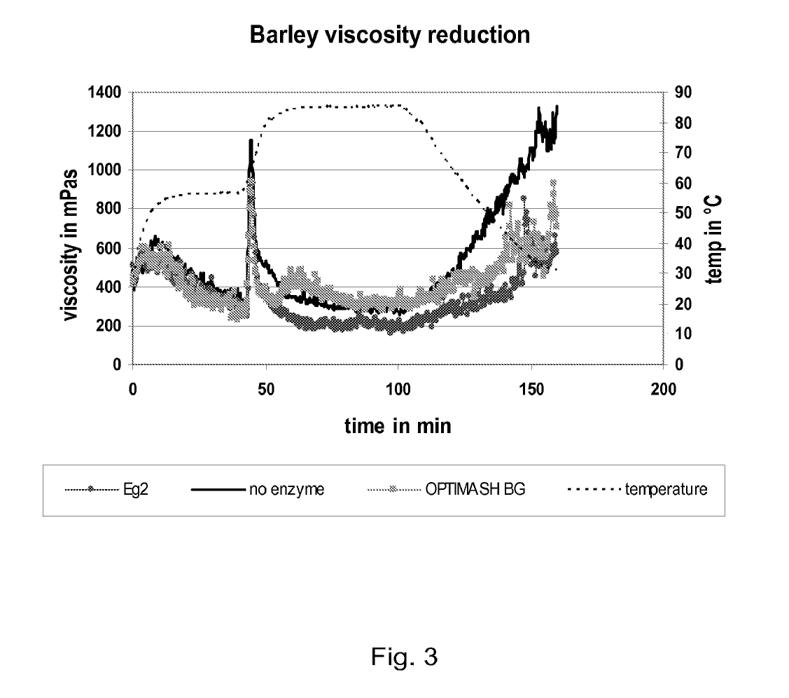

As shown in FIG. 4, both the lower dose (0.0164 kg/MT) and the higher dose (0.05 kg/MT) of EG II were more effective than equivalent doses of OPTIMASH™ BG in reducing the viscosity of the slurry when the enzymes were added during slurry make-up. The lower dose of EG II was particularly more effective than the equivalent dose of OPTIMASH™ BG.

The lower dose of EG II was also more effective than an equivalent dose of OPTIMASH™ BG in reducing the viscosity of the slurry when the enzymes were added during slurry make-up and in the third liquefaction although the use of a higher dose of OPTIMASH™ BG appeared to off-set this effect. These results demonstrate that the EG II alone can be as effective, if not more effective, than a mixed cellulase composition in reducing the viscosity of a barley slurry when added during slurry make-up, with out without additional enzyme being added in third liquefaction.

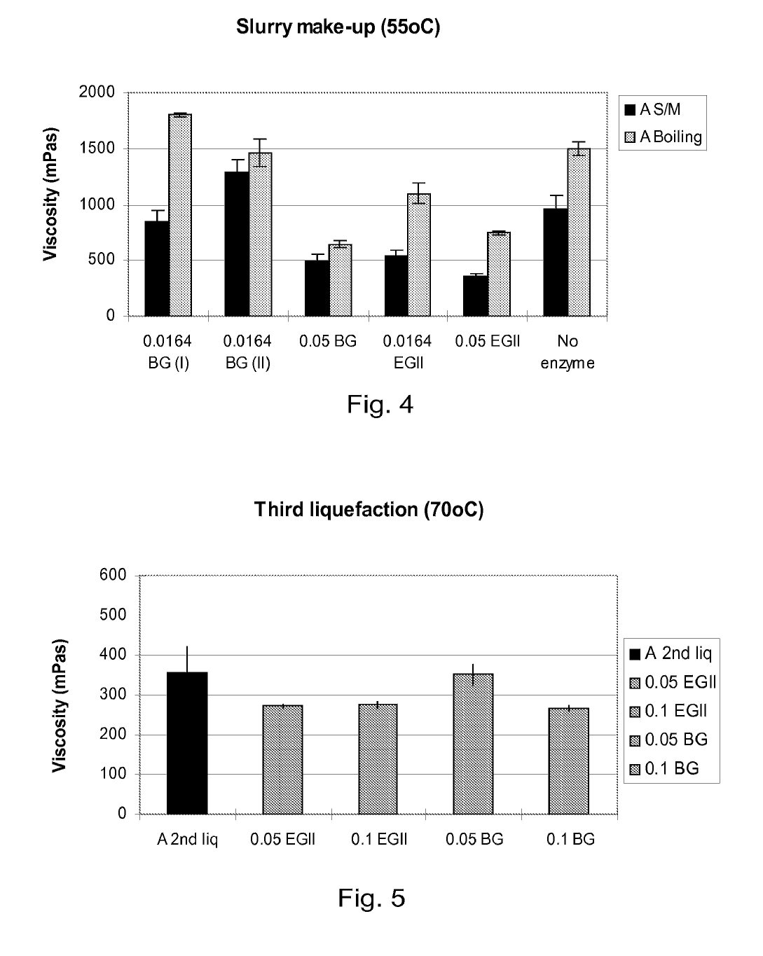

FIG. 5 shows a variation of the experiment wherein equivalent amounts of EG II or OPTIMASH™ BG were added to the slurry only in the third liquefaction step. At the lower dose (0.0164 kg/MT), EG II was more effective than OPTIMASH™ BG in reducing the viscosity of the slurry compared to the viscosity after the secondary liquefaction step. As above, the use of a higher dose of OPTIMASH™ BG appeared to off-set this effect. These results demonstrated that EG II alone can be as effective, if not more effective, than a mixed cellulase composition in reducing the viscosity of a barley slurry when added before the third liquefaction step.

### Example 4

### Activity of EG II Under Different pH and Temperature Conditions

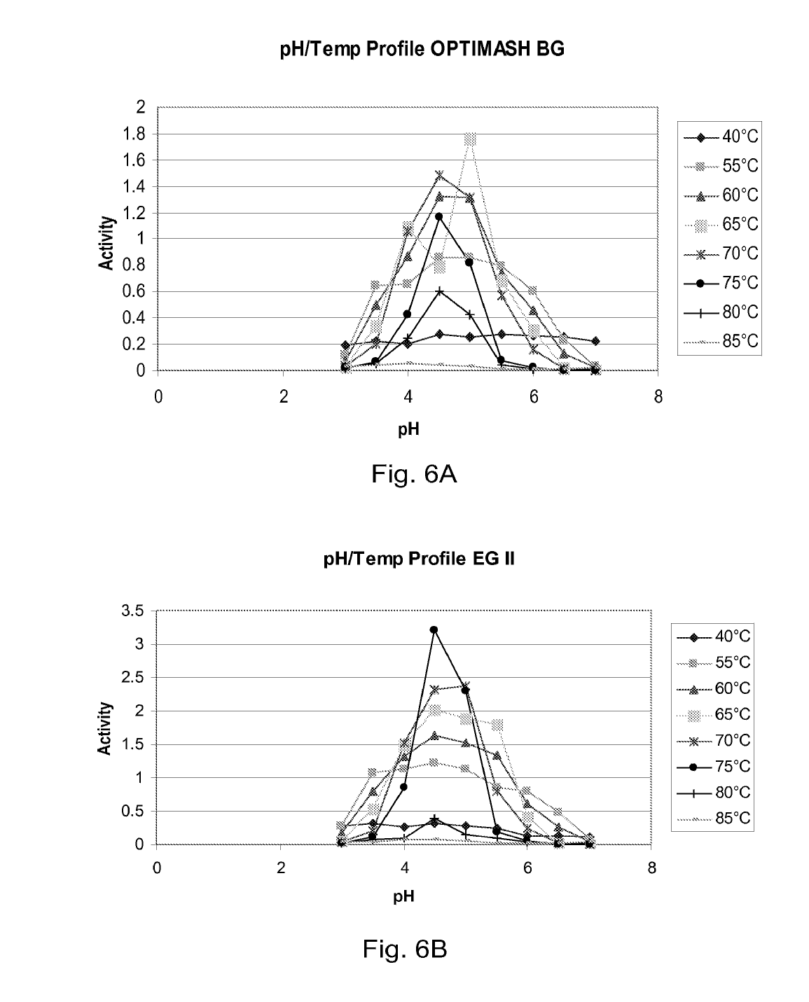

To determine whether EG II and OPTIMASH™ BG have different pH and/or temperature optima for liquefaction, the activity of each enzyme composition was tested against an artificial substrate [i.e., Celluzyme (azurine dye crosslinked to hydroxyethyl cellulose), commercially available from Megazyme International Ireland Ltd. (Wicklow, IR)] under different conditions. The pH/temperature profile of OPTIMASH™ BG is shown in FIG. 6A and the pH/temperature profile of EG II is shown in FIG. 6B. EG II was active over a broader pH range and at a higher temperatures than OPTIMASH™ BG, although the pH optimum narrowed at higher temperatures. The performance of EG II was best at 55-65° C.

These results suggest that EG II alone can be used over a broader pH range and at a higher temperature than OPTIMASH™ BG.

### Example 5

### Viscosity Reduction Using EG I, EG II, or EG III Compositions

To determine if other EG-cellulase compositions were able to liquify barley slurries in the absence of or CBH-type cellulases, BG-type cellulases, and xylanases, experiments similar to those described, above, were performed using EG I and EG III.

EGIII was produced essentially as in Example 1, using the same host strain of T. reesei transformed with an expression vector encoding the Trichoderma EG III polypeptide shown in FIG. 10B (SEQ ID NO: 4). EG I was produced in a similar manner using a host strain of T. reesei transformed with an expression vector encoding the Trichoderma EG I polypeptide shown in FIG. 10A (SEQ ID NO: 3). However, the host strain used to express EG I was not deleted for EG II, meaning that both EG I and EG II were expressed. CBH-type cellulases, BG-type cellulases, and xylanases were not expressed by this host strain, thus the only cellulases produced were of the EG-type.

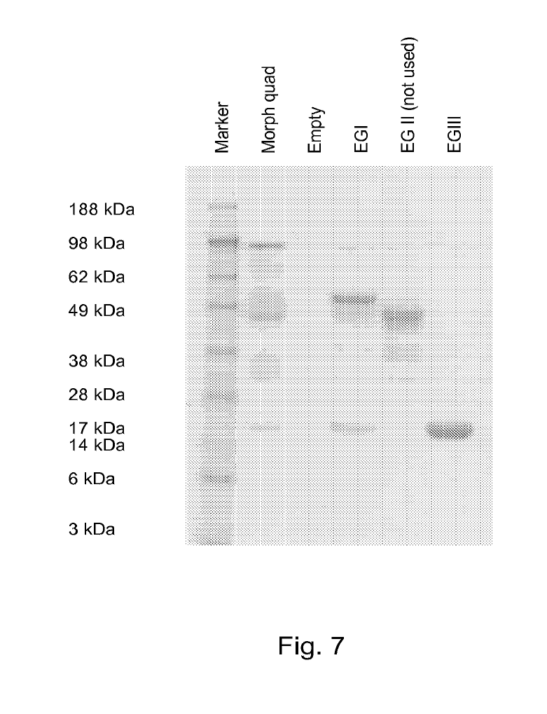

A Coomassie-stained SDS-PAGE gel showing the purity of the EG I and EG III preparations is shown in FIG. 7 (lane 1: protein marker, lane 2: Morph quad strain used for overexpression of EG II and III, lane 3: empty, lane 4: EG I (purity 77%), lane 5: a less pure EG II preparation (not used in any of the experiments), lane 6: EG III (purity 99%)).

200 grams of 25% DS slurry was prepared from barley (#2007116 milled at 10,000 rpm, with a 1 mm orafice size) with 50/50% demi/tap water. The native pH of the slurry was 5.6 without pH adjustment. 100 grams of the slurry was eventually added to the Haake viscotester for measurements.

A dosage of 0.1 kg/MT Spezyme Alpha™ (i.e., alpha-amylase from Geobacillus stearothermophilus having the substitution S242Q; Danisco, Genencor Division, Palo Alto, Calif., USA) was used in combination with EG I, EG II, or EG III preparations at equal EG dosages. A roughly equivalent amount of total protein from the expression strain (not harboring an EG-encoding gene) was used as a control. Following pretreatment of the slurry for 40 min. at 55° C., the temperature was increased to 85° C. to induce the solubilization of beta-glucan. After the 85° C. treatment, the slurry was cooled at 30° C. for 60 min., during which time residual betaglucan formed a gel, the viscosity of which correlated with the viscosity reducing effect of the enzymes added to the slurry with a shear rate of 50.0 l/S using an FL10 rotor-shaped spindle.

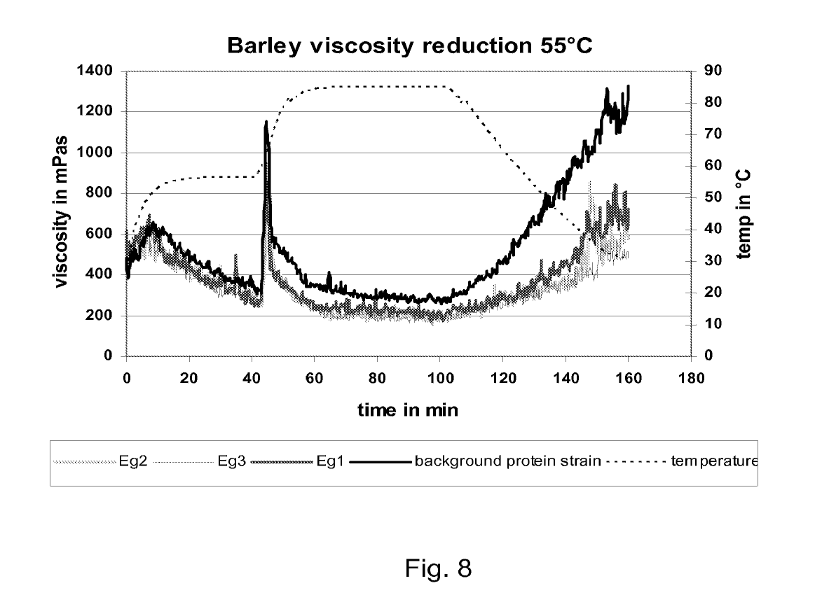

Viscosity was measured every 20 seconds during the experiment. As shown in FIG. 8, EG I, EG II, and EG III compositions were similarly effective in reducing the viscosity of the barley slurry. These results demonstrate that EG-type cellulases, in the absence of CBH-type cellulases, BG-type cellulases, and xylanases, are effective in reducing the viscosity of slurries in which betaglucan is primarily responsible for viscosity.

All patents and publications mentioned in the specification are indicative of the levels of those skilled in the art to which the invention pertains, and are incorporated by reference. Those of skill in the art readily appreciate that the present invention is well adapted to carry out the objects and obtain the ends and advantages mentioned, as well as those inherent therein. The compositions and methods described herein are representative of preferred embodiments, are exemplary, and are not intended as limitations on the scope of the invention. It is readily apparent to one skilled in the art that varying substitutions and modifications may be made to the invention disclosed herein without departing from the scope and spirit of the invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not excised material is specifically recited herein.

1. A method for reducing the viscosity of a plant material slurry comprising barley or oats, comprising adding to the slurry a composition comprising an isolated endoglucanase (EG) cellulase.

2. The method of claim 1, wherein the composition is substantially free of other cellulases.

3. The method of claim 2, wherein an addition cellulase is separately added to the slurry but is not required to reduce the viscosity of the slurry.

4-5. (canceled)

6. The method of claim 1, wherein an additional cellulase is separately added to the slurry but is not required to reduce the viscosity of the slurry.

7. The method of claim 1, wherein the EG cellulase is expressed in a filamentous fungus.

8. The method of claim 7, wherein the EG cellulase is expressed in Trichoderma reesei.

9. The method of claim 8, wherein the EG cellulase is expressed under control of the cbh1 promoter.

10. The method of claim 1, wherein the EG cellulase is purified to at least 70% of total protein in the composition.

11. The method of claim 1, wherein the EG cellulase is purified to at least 80% of total protein in the composition.

12. The method of claim 1, wherein the EG cellulase is purified to at least 90% of total protein in the composition.

13. The method of claim 1, wherein the EG cellulase is purified to at least 95% of total protein in the composition.

14. The method of claim 1, wherein the EG cellulase is purified to at least 97% of total protein in the composition.

15. The method of claim 1, wherein the EG cellulase is a Trichoderma reesei (Hypocrea jecorina) EG cellulase.

16. The method of claim 1, wherein the reduction in viscosity as a result of adding the EG cellulase is at least equivalent to a reduction in viscosity as a result of adding a mixture of cellulases wherein EG is a component.

17. The method of claim 1, wherein the EG cellulase is selected from the group consisting of EG I, EG II, and EG III.

18. The method of claim 1, wherein the EG cellulase is EG II.

19. The method of claim 18, wherein reducing the viscosity of the slurry is performed at a temperature greater than about 65° C.

20. The method of claim 1, wherein EG is added to the slurry prior to boiling.

21. The method of claim 1, wherein the addition of EG follows boiling the slurry.

22. The method of claim 1, wherein viscosity in the plant material slurry is primarily due to the presence of betaglucan.

23. The method of claim 1, wherein the plant material is from oats.

24. The method of claim 1, wherein the plant material is from barley.

25. A single-cellulase composition comprising EG cellulase, wherein the EG cellulase is expressed as a secreted polypeptide in filamentous fungi wherein other cellulase genes are deleted or disrupted.

26. (canceled)

27. The composition of claim 25, wherein the EG cellulase is selected from the group consisting of EG I, EG II, and EG III.

28. The composition of claim 25, wherein the EG cellulase is EG II.

29. The composition of claim 25, wherein the composition does not comprise a CBH cellulase, a BG cellulase, and/or a xylanase.

30. A cellulase composition according to claim 25 comprising one or more EG cellulases, said composition lacking a CBH cellulase, a BG cellulase, and/or a xylanase.


SEQUENCE LISTING (4 sequences)

SEQ ID NO: 1 - protein, 418 aa, Artificial Sequence
  Synthetic polypeptide variant EGII
MNKSVAPLLLAASILYGGAAAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNDDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTGSGNSWTDTSLVSSCLARK

SEQ ID NO: 2 - DNA, 1436 bp, Artificial Sequence
  Synthetic nucleotide sequence of variant Trichoderma EGII gene
atgaacaagtccgtggctccattgctgcttgcagcgtccatactatatggcggcgccgctgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagagtgagtacccttgtttcctggtgttgctggctgaaaagttgggcgggtatacagcgatgcggactgcaagaacaccgccggtccgccaccatcaagatgtgggtggtaagcggcggtgttttgtacaactacctgacagctcactcaggaactgagaattaatggaagtcttgttacagtggcacttgcgttacctcgaaggtttatcctccgttgaagaacttcaccggctcaaacaactaccccgatggcatcggccagatgcagcacttcgtcaacgacgacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactggcagtggtaactcatggacggacacatccttggtcagctcgtgtctcgcaagaaagtag

SEQ ID NO: 3 - protein, 459 aa, Trichoderma sp.
  misc_feature: EGI polypeptide
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 4 - protein, 234 aa, Trichoderma sp.
  misc_feature: EGIII polypeptide
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN